(19) United States
(12) Patent Application Publication
(10) Pub. No.: US 2003/0118663 A1
(43) Pub. Date: Jun. 26, 2003

(54) Method and composition for treating diabetes

(76) Inventor: Ching-Ming Chang, Taichung (TW)

Correspondence Address: Chang, Ching-Ming, P. O. Box 55-846, Taipei, 104, TW

(21) Appl. No.: 10/033,472

(22) Filed: Dec. 24, 2001

(51) Int. Cl. A61K 35/78; A61K 35/407

(52) U.S. Cl. 424/553; 424/757; 424/774; 424/777

(57) ABSTRACT

A therapeutic composition for treating diabetes includes mixing of the following ingredients:

a) a protein-containing material selected from the group consisting of: Mori Folium, soybean and dried powder of Bonito or Bonito liver;

b) a polysaccharide-containing material selected from the group consisting of: Lycii Fructus and Mori Folium; and

c) a carrier selected from yeast for efficiently decreasing the blood sugar level of a diabetic patient.

### BACKGROUND OF THE INVENTION

A diabetes disease can be detected by analyzing blood samples for elevated blood sugar concentrations. For instance, if a patient has a blood sugar level higher than 140 mg/dl before meal; and a blood sugar level higher than 200 mg/dl after meal; with excessive thirst and urination; showing his or her diabetic symptoms.

For treating the diabetes, insulin injection or oral antidiabetic drugs can be taken in order to well control the blood sugar levels. However, the injection or oral dosage of conventional antidiabetic medicines may cause side effects, for instance, serious low blood sugar level to easily affect brain such as dizziness or even unconsciousness, causing skin irritation or itching, and having gastrointestinal disorder such as vomit.

The present inventor has found the drawbacks of conventional treatments for curing diabetes, and invented the present method and composition for treating diabetes without side effect.

### SUMMARY OF THE INVENTION

The object of the present invention is to provide a method by administering a therapeutic composition for treating diabetes including mixing of the following ingredients:

a) a protein-containing material selected from the group consisting of: Mori Folium, soybean and dried Bonito liver or Bonito;

b) a polysaccharide-containing material selected from the group consisting of: Lycii Fructus and Mori Folium; and

c) a carrier selected from yeast for efficiently decreasing the blood sugar level of a diabetic patient.

### DETAILED DESCRIPTION

The present invention comprises the formulation of a therapeutic composition for treating diabetes by mixing the following ingredients:

a) a protein-containing material selected from the group consisting of: Mori Folium (leaves of mulberry tree), soybean and powder of dried liver of Bonito;

b) a polysaccharide-containing material selected from the group consisting of: Lycii Fructus (dried fruits of Chinese wolfberry or Lycium chinensis Mill.) and Mori Folium; and

c) a carrier selected from yeast (preferably manufactured from molasses of sugar industry).

The present invention comprises a method by orally administering the above-mentioned composition which can be prepared into capsules.

The protein (as contained in Mori Folium, soybean and dried bonito (liver) powder) of the present invention will stimulate the secretion of insulin to thereby decrease the concentration of blood sugar in the patient's blood.

The polysaccharide (as contained in Lycii Fructus and Mori Folium) in the present invention may be carried or transferred into the cells or liver to be stored as fuel, thereby providing enough nutrition for a diabetes patient. Since the hydrolysis reaction from polysaccharide to mono saccharide (sugar) will take a longer time, the sugar concentration in the patient's blood will be decreased, thereby being beneficial to the diabetes patient. Moreover, the protein and polysaccharide may interact to synergetically stimulate the secretion of insulin to decrease the blood sugar concentration for treating diabetes.

The carrier (yeast) plays an important role by carrying the protein and polysaccharide as above-mentioned into the cells in order to decrease the blood sugar level.

Accordingly, the blood sugar concentration of a diabetes patient will be efficiently decreased to help treat the diabetic disease.

### EXAMPLE 1

The following ingredients are ground, mixed and filled into capsules each containing 500 mg:

a) Mori Folium, 20˜50% (by weight);

b) Lycii Fructus, 10˜45% (by weight);

c) Soybean, 10˜30% (by weight); and

d) Yeast, 10˜30% (by weight).

### EXAMPLE 2

The formulation of the present invention as prepared by Example 1 is taken for testing in this example. A male patient, 56 years of age, is voluntarily invited for testing the effect of the present invention for two weeks. In the first week, he is orally dosed with one capsule per meal and 3 capsules daily. In the second week, the oral dosage is increased to be 6 capsules per day. His testing result before and after the treatment of the capsules are summarized as follows:

a. The blood sugar level before treatment of the present invention is checked as: 180 mg/dl before meal; and 240 mg/dl, 2 hours after meal.

b. The blood sugar level after treatment of the formulation (capsules) of the present invention:

70 mg/dl before meal; and

80 mg/dl, 2 hours after meal.

It indicates that the dosage of the capsules prepared by the present invention can efficiently decrease the blood sugar level, thereby being beneficial for helping curing a diabetes disease.

The composition of the present invention is dissolved in water to prepare a 10% (weight) injection solution to be injected into a rat (20 g body weight) everyday. The daily injection is continuously conducted for 21 days, showing no damage to the rat's internal organs. [Note: The quantity of the present invention applied to rat is about 60 times provided for the patient.]

So, the present invention has no side effect of toxicology.

The present invention provides a formulation of Chinese Medicine having plural ingredients mixed for synergetically treating diabetes. Since the ingredients are selected from natural sources, they cause no side effect for human being. So, it may serve as an aid for treating diabetes to prevent the side effect as caused from a conventional antidiabatic drug made of synthetic chemicals.

The present invention may be modified without departing from the spirit and scope of the present invention. Besides the ingredients as aforementioned, the Mori Folium may, for instance, be substituted with powder of dried liver of bonito or dried bonito powder. All ingredients should be well dried, ground and mixed.

I claim:

1. A method for treating diabetes comprising the administering of a therapeutic composition comprising mixing of the following ingredients:
a) a protein-containing material selected from the group consisting of: Mori Folium, soybean and powder of dried Bonito liver or Bonito;
b) a polysaccharide-containing material selected from the group consisting of: Lycii Fructus and Mori Folium; and
c) a carrier selected from yeast for efficiently decreasing the blood sugar level of a diabetic patient.

2. A therapeutic composition for the treatment of diabetes comprising mixing of the following ingredients:
a) a protein-containing material selected from the group consisting of: Mori Folium, soybean and powder of dried Bonito liver;
b) a polysaccharide-containing material selected from the group consisting of: Lycii Fructus and Mori Folium; and
c) a carrier selected from yeast for efficiently decreasing the blood sugar level of a diabetic patient.

3. A therapeutic composition according to claim 2, wherein said yeast is made from molasses.
